(19) United States
(12) Patent Application Publication
(10) Pub. No.: US 2004/0241667 A1
(43) Pub. Date: Dec. 2, 2004

(54) Pulse-jet ejection head diagnostic system

(76) Inventors: William G. Chesk, San Jose, CA (US); Donald J. Schremp, San Jose, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/452,800

(22) Filed: May 30, 2003

(51) Int. Cl. C12Q 1/68; G01N 1/00

(52) U.S. Cl. 435/6; 422/100

(57) ABSTRACT

A pulse jet head diagnostic system is provided for a pulse jet biopolymeric array fabrication device. The system and associated method enable electrically monitoring signals produced or output by a driver for effecting droplet ejection from an ejection head spaced from a substrate surface and during movement relative to the substrate surface onto the substrate surface. Real-time errors in performance may be reported, or results simply recorded for post-fabrication quality control. Quality control checking may be performed in-process during array fabrication or the systems described may be used as off-line diagnostic tools for ejection elements and/or fire pulse aspects of an array fabrication system. A computer program product that can execute the foregoing methodology and/or provide results of the same is also provided.

### FIELD OF THE INVENTION

This invention relates to arrays, such as polynucleotide or other biopolymer arrays (for example, DNA arrays), which are useful in diagnostic, screening, gene expression analysis, and other applications. More particularly, the invention relates to array production, especially with respect to diagnostic systems for quality control of pulse jet ejection heads and associated circuitry used in producing such arrays.

### BACKGROUND OF THE INVENTION

Array assays between surface bound binding agents or probes and target molecules in solution may be used to detect the presence of particular analytes or biopolymers in the solution. The surface-bound probes may be oligonucleotides, peptides, polypeptides, proteins, antibodies or other molecules capable of binding with target biomolecules in the solution. Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in an array on a substrate such as a glass substrate or the like. A solution containing target molecules (“targets”) that bind with the attached probes is placed in contact with the bound probes under conditions sufficient to promote binding of targets in the solution to the complementary probes on the substrate to produce a binding complex that is bound to the surface of the substrate. The pattern of binding by target molecules to probe features or spots on the substrate produces a pattern, i.e., a binding complex pattern, on the surface of the substrate that is detected. This detection of binding complexes provides desired information about the target biomolecules in the solution.

The binding complexes may be detected by reading or scanning the array with, for example, optical means—although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

Biopolymer arrays can be fabricated by depositing previously obtained biopolymers (such as from synthesis or natural sources) onto a substrate, or by in situ synthesis methods. Methods of depositing obtained biopolymers include depositing drops onto a substrate from dispensers such as pin or capillaries (such as described in U.S. Pat. No. 5,807,522) or such as pulse jets (such as a piezoelectric inkjet head, as described in PCT publications WO 95/25116 and WO 98/41531, and elsewhere). The substrate is coated with a suitable linking layer prior to deposition, such as with polylysine or other suitable coatings as described, for example, in U.S. Pat. No. 6,077,674 and the references cited therein. Additional references that describe aspects of our in situ and deposition systems for array fabrication include U.S. Pat. No. 6,242,266, U.S. Pat. No. 6,232,072, U.S. Pat. No. 6,180,351, U.S. Pat. No. 6,171,797, U.S. Pat. No. 6,323,043 and U.S. patent application Ser. No. 09/302,898 filed Apr. 30, 1999 by Caren, et al. The disclosures of all of the above references concerning array fabrication being incorporated herein by reference in their entireties.

In array fabrication, the quantities of polynucleotide available, whether by deposition of previously obtained polynucleotides or by in situ synthesis, are usually very small and expensive. Additionally, sample quantities available for testing are usually also very small and it is therefore desirable to simultaneously test the same sample against a large number of different probes on an array. These conditions require use of arrays with large numbers of very small, closely spaced features.

It is very important in such arrays that features actually be present, that they are put down accurately in the desired target pattern, are of the correct size, and that the nucleic acid or other chemical moiety is uniformly coated within the feature. Failure to meet such quality requirements can have serious consequences to diagnostic, screening, gene expression analysis or other purposes for which the array is being used. However, for economical mass production of arrays with many features it is desirable that they can be fabricated in a short time while maintaining quality.

Various systems have been developed to quickly and accurately produce arrays using pulse jet printheads. A very successful example is presented in the assignee's U.S. patent application Ser. No. 10/206,446, entitled “Fabricating Arrays with Drop Velocity Control.” That system is described as optionally employing a mode of quality control that relies on optical detection/inspection of the printed product. Such discussion is also provided herein in the Detailed Description below in that it may offer supplemental features to the present invention. Yet another known mode of quality control employs checking the data signal applied to a printhead driver, which driver is intended to actuate printhead ejection element(s). This approach simply employs shift register verification of the subject data.

In contrast to either approach, a primary features of present invention offers systems in which the electrical signals actually effecting printhead firing are detected (not just the outcome of a printed product as in the first quality control system example above, or the intended firing pattern for printed product). This invention tests actual driver operation. Such a system can be produced in a very cost effective manner, potentially offers certain diagnostic capabilities previously only possible by virtue of manual inspection of a printhead and/or its driver, and may improve efficiency in quality control (either because it offers real-time capability or simply by virtue of the speed at which printing-related data that is generated can be electronically verified). Accordingly, whether it is provided as supplemental to existing system or as the sole means of quality control, the invention meets the need for continuing improvement in providing quality control in the demanding art of array/microarray fabrication.

### SUMMARY OF THE INVENTION

The present invention then, provides in one aspect a method for fabricating a chemical (e.g., biopolymer) array with multiple features. The method includes directing ejection of reagent drops from a pulse jet print or ejection head spaced from a substrate onto the substrate surface while verifying that such ejection actually occurred by checking for electrical signals indicative of the same. Of course, the print head and the substrate will move relative each other (i.e., one or the other or both may be moved) during this procedure in order to lay down the desired array pattern. The array may be a biopolymer array (e.g., a polynucleotide array), in which case at least some of the ejected drops comprise the biopolymers or their precursor units (e.g., monomer units of the biopolymers).

One of at least two circuit layouts may be employed in order to achieve the subject verification or quality control features for driver and/or ejection element function. The type of circuit depends on the manner of ejection or pulse jet head construction. Either piezo-based or resistor-based thermal ink jet heads may be employed in the invention. An output from a gate array connected to a printhead circuit, preferably via a voltage divider, provides indication of whether a particular ejection element associated with a jet or nozzle fired (as expected) in order to deliver the print medium being laid down. Another quality control or diagnostic circuit may also, or alternatively, be provided to test for basic fire pulse function for a print head and/or verify pulse amplitude.

The present invention includes associated quality control methodology as well as the hardware configured to carry out the same. Such methodology may be carried our as a “real-time” quality control check.

As to the hardware that may be employed for implementing the invention, the apparatus includes a substrate station to retain a substrate thereon. An ejection head is provided facing and spaced from a retained substrate. A transport system moves one of the head and retained substrate relative to the other. A control unit controls the ejection head and transport system so as to execute a method of the present invention. For example, the control unit ejects drops from the ejection head while the head is spaced apart from a retained substrate surface and during movement relative to the substrate surface, onto the substrate surface while performing quality control.

The present invention also provides a computer program product. The computer program product may comprise a computer readable medium which, when loaded into a programmable computer, executes a method described herein. Still further, the product may comprise quality control results associated with a particular fabricated array chip/substrate. Such information may be particularly useful where off-line quality control checking is performed instead of real-time analysis.

In short, the present invention includes the subject methodology, programming defining the same, including such algorithms as applied to the array fabrication and quality control solution taught herein, hardware configured to run according to the methodology, and results or data produced according to the teachings of the present invention.

### BRIEF DESCRIPTION OF THE DRAWINGS

Each of the figures diagrammatically illustrates aspects of the invention. To facilitate understanding, the same reference numerals have been used (where practical) to designate similar elements that are common to the figures.

FIG. 1 shows an array fabrication system as may be used in the present invention.

FIGS. 2A and 2B shows diagnostic circuits as may be used in connection with the system of FIG. 1 or another printing system.

FIG. 3 shows another diagnostic circuit as may be employed in the invention.

FIG. 4 is a top view of a packaged array that may be produced according to and used in connection with the present invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups. Biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (the disclosure of which is incorporated herein by reference), regardless of the source. An “oligonucleotide” generally refers to a nucleotide multimer/polymer) of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

An “array,” includes any one dimensional, two-dimensional or substantially two-dimensional (as well as a three-dimensional) arrangement of addressable regions bearing a particular chemical moiety or moieties (e.g., biopolymers such as polynucleotide sequences (nucleic acids), polypeptides (e.g., proteins), etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

The term “substrate” as used herein refers to a surface upon which marker molecules or probes, e.g., an array, may be adhered. Glass slides are the most common substrate for biochips, although fused silica, silicon, plastic and other materials are also suitable. The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage. “Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C. A “web” references a long continuous piece of substrate material having a length greater than a width. For example, the web length to width ratio may be at least 5/1, 10/1, 50/1, 100/1, 200/1, or 500/1, or even at least 1000/1. The substrate may be flexible (such as a flexible web). When the substrate is flexible, it may be of various lengths including at least 1 m, at least 2 m, or at least 5 m (or even at least 10 m). The term “rigid” is used herein to refer to a structure e.g., a bottom surface or a cover that does not readily bend without breakage, i.e., the structure is not flexible.

Any given substrate may carry one, two, four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other).

A “pulse jet” is a device that can dispense drops/droplets in the formation/fabrication of an array. Pulse jets operate by delivering a pulse of pressure (such as by a piezoelectric or thermoelectric element) to liquid adjacent an outlet or orifice such that a drop will be dispensed therefrom. When the arrangement, selection, and movement of “dispensers” is referenced herein, it will be understood that this refers to the point from which drops are dispensed from the dispensers (such as the outlet orifices or nozzles of pulse jets).

A “drop” in reference to the dispensed liquid does not imply any particular shape, for example a “drop” dispensed by a pulse jet only refers to the volume dispensed on a single activation. A drop that has contacted a substrate is often referred to as a “deposited drop” or “sessile drop” or the like, although sometimes it will be simply referenced as a drop when it is understood that it was previously deposited. The terms “fluid” and “liquid” are used synonymously herein in reference to a solution or other flowable and/or printable medium. Detecting a drop “at” a location, includes the drop being detected while it is traveling between a dispenser and that location, or after it has contacted that location (and hence may no longer retain its original shape).

An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

A “scan region” refers to a contiguous (e.g., rectangular) area in which the array spots or features of interest, as defined above, are found. The scan region is that portion of the total area illuminated from which the resulting fluorescence, chemiluminescence, or other optical detection techniques is detected and recorded. For the purposes of this invention, the scan region includes the entire area of the slide scanned in each pass of the lens, between the first feature of interest, and the last feature of interest, even if there exist intervening areas that lack features of interest. The scan region does not, however, include “border regions” or “borders” of the array substrate/slide adjacent slide edges and adjacent to but not including or covered by array features. Generally, any borders around the scan region are less than about 5-15 mm and can be as little as 1 mm, or even smaller, if the mechanical design of the slide holder permits it. It is often desirable to lay down features as close to the edge of the substrate as possible so as to maximize the number of different probes that may be displayed on a given surface area. As such, in many array configurations, the width of a border, if present, between the scanned arrays and the slide edge does not exceed about 20 mm, usually does not exceed about 10 mm and more usually does not exceed about 5 mm. “Lens position” refers to the relative distance between the lens or optical objective(s) of a scanner and a caddy carrying a slide and/or the slide or array itself.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Furthermore, the words such as “top”, “upper”, and “lower” are used in a relative sense only. Also, when one thing is “moved”, “moving”, “re-positioned”, “scanned”, or the like, with respect to another, this implies relative motion only such that either thing or both might actually be moved in relation to the other. For example, when dispensers are “moved” relative to a substrate, either one of the dispensers or substrate may actually be put into motion by the transport system while the other is held still, or both may be put into motion.

### DETAILED DESCRIPTION OF THE INVENTION

In describing the invention in greater detail than provided in the Summary and as informed by the Background and Definitions provided above, the subject program or process aspects of the invention are first described. Next, exemplary array fabrication hardware is described, including invention-specific hardware aspects of the same. This discussion is followed by a brief discussion of methods of using an array including which scanners may be used, and kits for use with product produced according to the invention.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element (as indicated by use of any permissive term such as the words “may,” “might,” “possible,” etc.). Accordingly, this statement is intended to serve as antecedent basis for use of such exclusive terminology as “solely,” “only” and the like in connection with the recitation of claim elements, or use of a “negative” limitation therewith.

Methodology/Programming

As summarized above, the present invention involves software and hardware associated with biopolymer array production—especially in connection with pulse-jet delivery of such material. Programming embodying the methodology may be loaded onto array production equipment, or the system may be preprogrammed to run with the same. The programming can be recorded on computer readable media, (e.g., any medium that can be read and accessed directly by a computer). Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information.

As to the subject methodology itself, it provides a method for checking the data or signal being actually applied to a particular ejection element of a print or ejection head. Generally, the ejection head will include a plurality of such elements (e.g., 128 piezo elements) with associated nozzles. If an electrical failure occurs in one of the driver and/or the elements directly responsible for ejecting a droplet from a printhead, the present invention can detect the same, even in real-time. Such detection and associated product quality control is accomplished without need to make visual reference to, or by, direct optical examination of the printed product.

More specifically, specialized circuitry is used for generating performance data to be checked against a data pattern intended for producing a desired array layout. Performance data generated for each print operation (e.g., each piezo or thermal element firing cycle) intended in array production may be used during the array printing process for immediate verification. Alternatively, the data generated may be stored for subsequent analysis. In either case, a complete verification of the electronic path involved in the printing process can be accomplished.

The routine and associated hardware may offer functionality in terms of a means of quality control for an ejection head, or as a diagnostic tool useful for troubleshooting suspect ejection elements. The methodology also contemplates programming to direct action during of the array fabrication based on quality control determinations. As an example of directing such action, the system, upon detecting a misfire, could reconfigure printing nozzles and substrate positioning to accomplish the desired printing job by utilizing the remaining functioning nozzles. In this way, a real time detected malfunction could be worked around dynamically without stopping the process, enhancing production quality and throughput. In that the remainder of the text below focuses on other aspects of the invention, this fact is not intended to vitiate the utility and benefits in such other applications of the invention.

Methods of the invention involve monitoring the effect of a firing pulse voltage applied to individual ejection elements of a print head. The methodology may be implemented utilizing piezo or resistance-based print heads in at least two primary ways. Each manner noted depends on the operational design of the print/ejection head employed, specifically with respect to the manner in which it's the ejections elements are intended to be fired or activated.

A first contemplated approach involves a printhead configured for ejection upon a driver effecting a grounded state for each firing element when a common firing pulse is applied. The second approach involves a print head in which its elements are always grounded (possibly by a common ground connection) and firing is effected by application of individual firing pulses to each ejection element.

In the first approach, grounding an element by a driver provides a path across the element for current to flow therethrough (momentarily, in the case of a piezo element). This action causes ejection element activation and fluid expulsion through the associated nozzle—assuming proper function of other hardware, mechanical and/or fluidic aspects of the system.

As such, a flow of current from a firing pulse source, through the ejection element, and to ground temporarily “shorts out” a quality control or detection circuit (provided between the ejection element and corresponding driver) having a higher resistance. In the case where the element will not be fired, the driver provides an open circuit to ground. Such a condition results in negligible current passing through the ejection element capacitance. Yet, it provides an amount of current traveling through the quality control circuit at a voltage that is sufficient to set a latch in logic provided.

The logic may be provided by a Field Programmable Gate Array, (hereinafter a “FPGA”). Such a device will be connected at its “set” inputs to individual ejection elements. Where the quality control circuit includes provision to set a plurality of internal latches corresponding to the number of elements as described, a no (or low) voltage signal will correspond to a reset (not set) latch. As noted above, a voltage applied (or that continues to be applied depending on the previous condition) will set (or retain a set condition for) the latch. Correct firing may be indicated as such by either a set or reset latch, depending on the data pattern. A firing error occurs when a nozzle does not fire when intended or if a nozzle fires when it is not supposed to.

After each fire pulse (an event that may be on the order of 5 μs or longer) and generating data indicative of whether printhead ejection should have occurred (by either setting a latch or not), latch shift register contents are shifted out of the same. The register is also cleared for the next fire pulse to ready the system for a subsequent print operation. The contents of the shift register are output to a suitable device (e.g., to a computer via a serial port) for verification. Such verification will generally occur by way of as a check against expected digital values corresponding to a desired print pattern.

Essentially, the FPGA circuit records which nozzles fire and which nozzles remain idle during a firing event by the set-reset latch states. This pattern is then shifted out of the FPGA as a binary string (1's and 0's) and compared to the programmed data pattern that initiated the firing event. The two patterns should match if no error occurred. An error will have occurred for every mismatch in the read back string.

As provided above, the data generation scheme offers a sort of “reverse” logic in the sense that a significant voltage applied to the quality control circuit (registering a one in logic), indicates a no-fire condition. When firing does take place, no significant voltage is applied to the quality control circuit (registering a zero in logic). Of course, the latch information can be flipped or transposed so zeros (0's) correspond to no fire/ejection and ones (1's) correspond to an ejection head element firing instead.

Regardless of the logic implementation in this regard, where comparison of generated data to expected layout information occurs after every fire pulse, it provides a means of accomplishing a “real-time” check of whether the intended array pattern data is being applied to substrate across from an ejection head. Alternatively, the quality control data can be saved and processed upon completion of printing an array or later. In this manner, data processing can occur off-line or by a separate system specifically intended to carry out quality control. Whether applied is a real-time manner or not, comparison of data generated by the quality control circuitry to expected values can also be used to isolate defective ejection and/or driver element(s).

The same benefits and use can be achieved in connection with the second type of print head noted above. Namely, in an ejection head where the ejection elements are always grounded and discrete firing pulses are provided to actuate the ejection elements, “real time” quality control is also possible.

In the case of such hardware, the selection of the printhead ejection element is done by the circuitry providing the fire pulse. The presence of the fire pulse is detected regardless of the presence (proper function) of the element (i.e., the ejection element does not short out the quality control circuit). Instead, the fire pulse appears at the element and the quality control circuit simultaneously. As such, the presence of the fire pulse sets the latch while the absence of the fire pulse does not set it, so the latch remains reset. Proper firing is defined as the presence of a fire pulse when a nozzle is to be fired as well as the absence of a fire pulse when a nozzle is not to be fired. The quality control circuit verifies that the mix of firing and non-firing nozzles corresponds to the programmed configuration for a given firing event. So, an error occurs if a fire pulse appears at a nozzle that is supposed to be idle or if no fire pulse appears when a nozzle is to be fired.

Both errors are caught and the information is contained in the data pattern that is captured in the FPGA every firing cycle. Yet, this hardware scheme does not allow verification of the electrical operation of the printhead (or even its presence) as the variation of the invention noted above. It is for this reason that the former setup and methodology may sometimes be preferred, even though the system just described offers certain utility. Namely, it still offers verification of the signal path up to the printhead element(s) and/or actual driver function, as in whether the intended firing signals are being received at this point.

With either print head setup and associated control, since the voltage typically applied to effect (e.g., piezo) actuation is relatively high (e.g., about 100V) and the acceptable input to a FPGA is relatively low (e.g., about 5V) a step-down in voltage is generally desired. For this purpose, the quality control circuit preferably includes a voltage divider through which current runs as appropriate to the methodology above before encountering the FPGA.

As for further specifics associated with hardware implementation of these aspects of the invention, these specifics are discussed below. First, a suitable array fabrication system is described. Then, details of the quality control circuitry associated with the above methodology is discussed. Finally, hardware aspects of the following are discussed.

Before such discussion is provided, however, certain other optional printer system diagnostic methodology is noted. Particularly, circuitry is provided in which it is possible to independently determine if a fire pulse is being sent to a print head. By providing a circuit adapted to receive a signal in the form of an attenuated fire pulse (where such attenuation is accomplished with a voltage divider), observation of this signal indicates an operative driver board. By converting the attenuated signal with an Analog-to-Digital converter (hereinafter an “ADC”) to a digital value, the amplitude of the analog signal can be checked by a computer or by a user to determine whether the driver is functioning properly for each pulse signal. Whether implemented to measure the amplitude of the input signal (particularly, as correlated to the fire pulse voltage) or instead with a simple logic switch (absent an ADC) that merely provides indication of firing such a circuit offers an additional automatic diagnostic or real-time quality control tool in the invention.

Array Fabrication Hardware

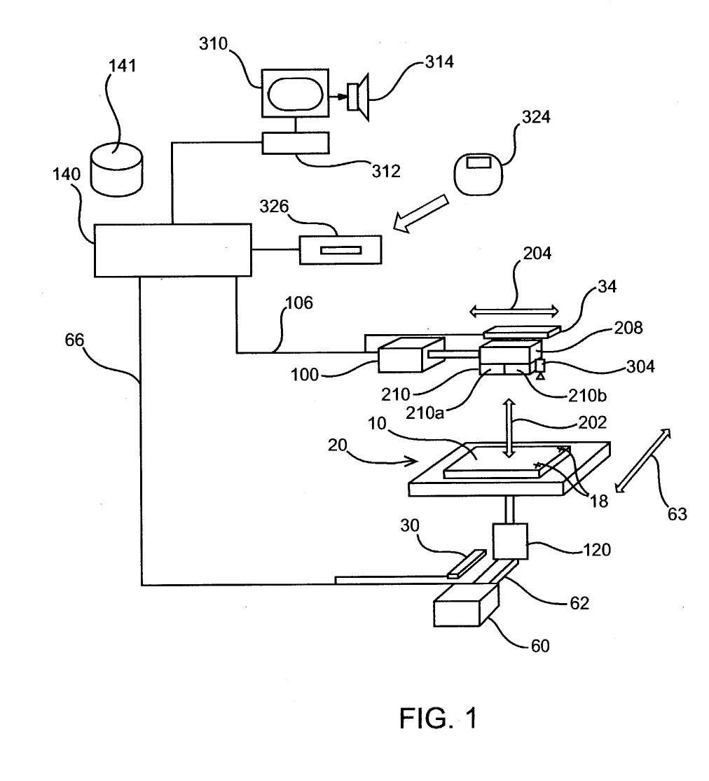

Referring to FIG. 1, an exemplary array fabrication system is provided. The apparatus includes a substrate station 20 on which a substrate 10 can be retained. Pins or similar means (not shown) can be provided on substrate station 20 by which to approximately align substrate 10 to a nominal position thereon. Substrate 10 may be retained on substrate station 20 simply by weight. However, more secure retention is provided by substrate station 20 including a vacuum chuck connected to a suitable vacuum source (not shown) to retain a substrate 10 without exerting too much pressure thereon, since substrate 10 is often made of glass or silica.

A movable print head system 210 (with two print or ejection heads 210a, 210b) is retained by a head retainer 208. Head system 210 can be positioned at any position facing a retained substrate 10, by means of a transport system. The transport system includes a carriage 62 connected to a first transporter 60 controlled by processor 140 through line 66, and a second transporter 100 controlled by processor 140 through line 106. Transporter 60 and carriage 62 are used to execute one axis positioning of station 20 (and hence mounted substrate 10) facing the dispensing head system 210, by moving it in the direction of nominal axis 63, while transporter 100 is used to provide adjustment of the position of head retainer 208 in a direction of nominal axis 204. In this manner, head system 210 can be scanned line by line, by scanning along a line over substrate 10 in the direction of axis 204 using transporter 100 while substrate 10 is stationary, while line by line movement of substrate 10 in a direction of axis 63 is provided by transporter 60 while head system 210 is stationary. Head system 210 may also optionally be moved in a vertical direction 202, by another suitable transporter (not shown). However, it will be appreciated that other scanning configurations could be used. Also, it will be appreciated that both transporters 60 and 100, or either one of them, with suitable construction, could be used to perform the foregoing scanning of head system 210 with respect to substrate 10. Thus, in reference to “positioning”, “moving”, or “displacing” or the like, one element (such as head system 210) in relation to another element (such as one of the stations 20 or substrate 10), it is to be understood that any required moving can be accomplished by moving either element or a combination of both of them.

An encoder 30 communicates with processor 140 to provide data on the exact location of substrate station 20 (and hence substrate 10 if positioned correctly on substrate station 20), while encoder 34 provides data on the exact location of holder 208 (and hence head system 210 if positioned correctly on holder 208). Any suitable encoder, such as an optical encoder, may be used which provides data on linear position. Angular positioning of substrate station 20 is provided by a transporter 120, which can rotate substrate station 20 about axis 202 under control of processor 140. Typically, substrate station 20 (and hence a mounted substrate) is rotated by transporter 120 under control of processor 140 in response to an observed angular position of substrate 10 as determined by processor 140 through viewing one or more fiducial marks on a retained substrate 10 (particularly fiducial marks 18) with a camera (such as camera 304). This rotation will continue until substrate 10 has reached a predetermined angular relationship with respect to dispensing head system 210. In the case of a square or rectangular substrate, the mounted substrate 10 will typically be rotated to align one edge (length or width) with the scan direction of head system 210 along axis 204.

Head system 210 may contain one or more (e.g., two or three) heads mounted on the same head retainer 208. Each such head may be the same in construction as a head type commonly used in an ink jet type of printer. Each ejector may be in the form of a piezoelectric crystal operating under control of processor 140 (although resistors for thermally activated ejectors could be used instead). The operative principle of the invention remains essentially the same in either case. Basically, the presence of an activating voltage (fire pulse) is detected using a voltage divider (two resistors) and a latch circuit.

Each orifice with its associated ejector and portion of the chamber, defines a corresponding pulse jet with the orifice acting as a nozzle. It will be appreciated that head system 210 could have any desired number of pulse jets (for example, at least fifty or at least one hundred pulse jets). In this manner, application of a single electric pulse to an ejector (however accomplished) causes a droplet to be dispensed from a corresponding orifice. Elements of each head can be adapted from commercially available piezoelectric and/or thermal inkjet print heads. One type of head and other suitable dispensing head designs are described in more detail in U.S. patent application entitled “A Multiple Reservoir Ink Jet Device for the Fabrication of Biomolecular Arrays” Ser. No. 09/150,507 filed Sep. 9, 1998.

As is well known in the ink jet print art, the amount of fluid that is expelled in a single activation event of a pulse jet, can be controlled by changing one or more of a number of parameters, including: the orifice diameter, the orifice length (thickness of the orifice member at the orifice), the size of the deposition chamber, and the size of the piezoelectric or heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, or may be more than about 10 m/s, and may be as great as about 20 m/s or greater.

The apparatus may further includes a sensor in the form of a camera 304, to monitor dispensers for errors (such as failure to dispense droplets) by monitoring for drops dispensed onto substrate 10 when required of a dispenser. Camera 304 can also image the structures on surface 11a. Camera 304 communicates with processor 140, and should have a resolution that provides a pixel size of about 1 to 100 micrometers and more typically about 4 to 20 micrometers or even 1 to 5 micrometers. Any suitable analog or digital image capture device (including a line by line scanner) can be used for such camera, although if an analog camera is used processor 140 should include a suitable analog/digital converter. A detailed arrangement and use of such a camera to monitor for dispenser errors, is described in U.S. Pat. No. 6,232,072, the disclosure of which is incorporated herein by reference. Particular observations techniques are described, for example, in co-pending U.S. patent application Ser. No. 09/302,898 filed Apr. 30, 1999 by Caren et al., assigned to the same assignee as the present application, the disclosure of which is incorporated herein by reference. Monitoring by such means can occur during formation of an array and the information used during fabrication of the remainder of that array or another array, or test-print patterns can be run before array fabrication.

Yet, no such provisions for optical monitoring need be made due to the features of the present invention. On one hand, they may provide certain desirable redundancy of offer supplemental features. Still, their omission may be desired from a basic cost-savings perspective.

Regardless, in the system shown in FIG. 1 a display 310, speaker 314, and a operator input device 312, may also provided. Operator input device 312 may, for example, be a keyboard, mouse, or the like. Processor 140 has access to a memory 141, and controls print head system 210 (specifically, the activation of the ejectors therein), operation of the transport system, operation of each jet in print head system 210, capture and evaluation of images from the camera 304, and operation display 310 and speaker 314. Memory 141 may be any suitable device in which processor 140 can store and retrieve data, such as magnetic, optical, or solid state storage devices (including magnetic or optical disks or tape or RAM, or any other suitable device, either fixed or portable).

Processor 140 may include a general purpose digital microprocessor suitably programmed from a computer readable medium carrying necessary program code, to execute all of the functions required of it as described below. It will be appreciated though, that when a “processor” such as processor 140 is referenced throughout this application, that such includes any hardware and/or software combination which will perform the required functions. Suitable programming can be provided remotely to processor 140, or previously saved in a computer program product such as memory 141 or some other portable or fixed computer readable storage medium using any of those devices mentioned below in connection with memory 141. For example, a magnetic or optical disk 324 may carry the programming, and can be read by disk reader 326.

As for the basic operation of the system in FIG. 1, first, it will be assumed that memory 141 holds a target drive pattern. This target drive pattern is the instructions for driving the apparatus components as required to form the target array (which includes target locations and dimension for each spot to form an array pattern) on substrate 10 and includes, for example, movement commands to transporters 60 and 100 as well as firing commands for each of the pulse jets in head system 210 coordinated with the movement of head system 210 and substrate 10, as well as instructions as to which polynucleotide precursor solution or activator solution is loaded in each pulse jet (i.e., that is, the “loading pattern”).

Such solutions may be provided to the different pulse jets through appropriate respective conduits (not shown) communicating between the head system 210 and respective reservoirs (not shown). An appropriate arrangement of the foregoing is disclosed, for example, in U.S. Pat. No. 6,372,483. The target drive pattern is based upon the target array pattern and can have either been input from an appropriate source (such as input device 312, a portable magnetic or optical medium, or from a remote server, any of which communicate with processor 140), or may have been determined by processor 140 based upon an input target array pattern (using any of the appropriate sources previously mentioned) and the previously known nominal operating parameters of the apparatus. Further, it will be assumed that drops of different biomonomer or biopolymer containing fluids (or other fluids) have been placed at respective regions of a loading station (not shown).

In instances where piezo-based printhead are used, note that in the target drive pattern the waveform supplied to each piezoelectric crystal in head system 210 determines the deformation of the crystal, which in turn determines the pressure pulse imparted on the fluid in the pulse jet. The velocity of the exiting drops can be adjusted by adjusting the amplitude of each pulse in the waveform. Adjustment of waveform to obtain velocity control is generally described in U.S. Pat. No. 6,402,282, European Patent Publication EP0721840A2, and U.S. patent application Ser. No. 10/206,446, the disclosures of which are incorporated herein by reference.

Substrate 10 is loaded onto substrate station 20, if not previously loaded, either manually by an operator, or optionally by a suitable automated driver (not shown) controlled, for example, by processor 140. The deposition sequence is then initiated to deposit the desired sequence of drops of nucleotide monomers (particular phosphoramidite monomers) or activator solution, onto the substrate according to the drive pattern. As already mentioned, in this sequence processor 140 will operate the apparatus according to the drive pattern, by causing the transport system to position head system 210 facing substrate station 20, and particularly the retained substrate 10, and with head system 210 at an appropriate distance from substrate 10. Processor 140 then causes the transport system to scan head system 210 across substrate 10 line by line (or in some other desired pattern), while co-coordinating activation of the ejectors in head system 210 so as to dispense droplets as described above. This may include the droplet deposition over multiple cycles as required by the in situ synthesis process. For the in situ process the substrate may be moved between cycles to a flood station for exposure of its entire surface to an oxidizing agent and deprotecting agent, in a known manner.

At this point the droplet dispensing sequence is complete and the arrays have been fabricated on surface. A final deprotection step may be required as is known.

Intermediate to such completion, however, where real-time quality control according to the present invention is being carried out, the process may be stopped short of completion. In which case the array may be discarded and another substrate provided for printing thereon. Alternatively, production may be stopped until detected errors are corrected. Still further, a data file may simply be generated to travel with the array and indicate tolerable print defects. Either of these later options (or others) may be employed where a quality control check is made after droplet deposition completion.

Quality Control Circuitry

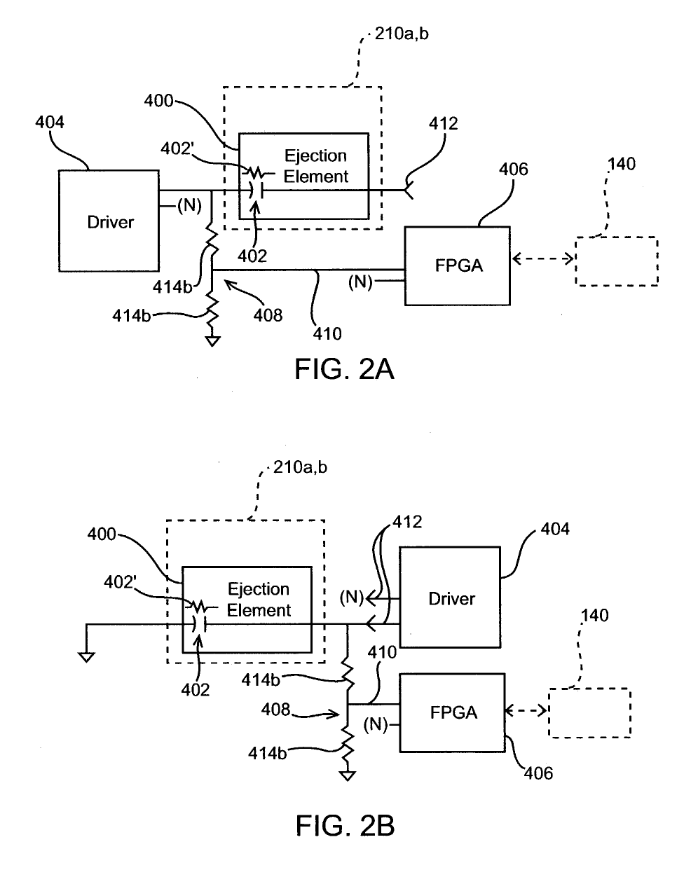

FIGS. 2A and 2B detail quality control circuitry as may be used in the subject methodology and in array fabrication with the exemplary system of FIG. 1 or otherwise. As applied to the noted fabrication system in FIG. 1, ejection elements 400 form part of the print or ejection head(s) 210a, 210b as indicated in each of FIGS. 2A and 2B.

Each element is shown as having a capacitance feature 402. As further commented upon below, a resistance or resistive feature 402′ may be substituted for the same when the printhead is one which is thermally activated. Additional common elements in the variations of the invention pictured in FIGS. 2A and 2B include a drivers 404 and FGPAs 406.

The system in FIG. 2A operates in the first mode discussed above where fluid ejection occurs upon the driver grounding individual ejection elements. The system in FIG. 2B operates by the second mode discussed above where print head ejection occurs upon delivery of individual fire pulses to each ejection element. In each of these subsystems, a designation (N) is provided where additional discrete ejection elements 400 and voltage dividers 408 are connected thereto. The voltage dividers include resistors 414a and 414b, each being easily selected to accounting for the internal resistance of FPGA 406, input voltage and FPGA voltage capacity). Stated otherwise, both FIGS. 2A and 2B show one piezo or resistor element corresponding to a single nozzle control in an ejection head. In the invention, there are N latch inputs 410 for N nozzles; also, there are N divider networks 408.

In the variation of the invention in FIG. 2A, a firing pulse 412 is provided as directed per controller 140 by a common connection to each of the N elements 400. Driver 404 individually pulls each such N elements 400 to ground to effect fluid ejection from the associated print head nozzle (not shown). In the variation of the invention in FIG. 2B, a common ground is provided for each of the N elements 400. Individual firing pulses 412 for each of the N elements 400 may be provided by a driver or otherwise to initiate printhead ejection. In any case, such activity is—again—directed by controller 140.

As described above, depending on the action and/or state of repair of the driver and the ejection element a signal corresponding to the activity printing (or diagnostic use of the system) may be obtained. Regardless of the methodology, the quality control circuits as referenced herein include at least the logic as properly posited to receive the intended signals. Usually, it also includes the associate voltage divider to make such input suitable. Still further, the circuit quality control referred to may be a single one such assemblies or the aggregation of each logic gate and/or voltage divider provided for a given printhead.

As discussed in the Methodology/Programming section above, data generated by the quality control circuitry (via FPGA 406) are preferably shifted out of the same for processing and/or storage. Storage may be provided by memory 141 in connection with controller 141 of the system presented in FIG. 1.

The invention also offers additional optional features for independently verifying the amplitude or simply the presence of a fire pulse applied to the printhead. In so doing, a complete verification and calibration system ejection head control can be provided. Furthermore, such features may be of use in addressing the shortcoming for the system in FIG. 2B noted above.

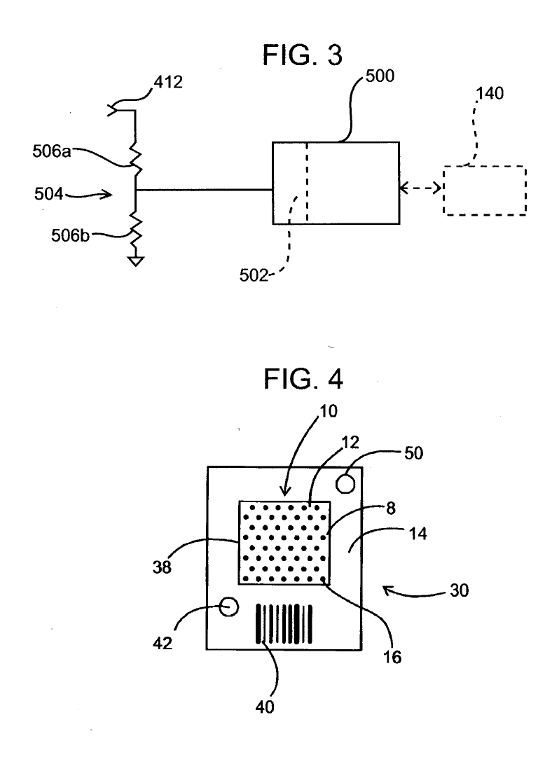

In the circuit shown in FIG. 3, a micro controller 500 is provided (though it may be part of controller 140). In either case, it is configured to include an ADC that measures a fire pulse 412 delivered from a driver (such as driver 404 in FIG. 2B) that is attenuated by a voltage divider 504 including appropriately selected resistor elements 506a, 506b. As indicated by dashed lines, a serial linking to a host processor 140 may be provided. The same may be true of the subsystems referenced in FIGS. 2A and 2B.

Utility

The subject biopolymer arrays produced in connection with the present invention find use in a variety applications, where such applications are generally analyte detection applications in which the presence of a particular analyte in a given sample is detected at least qualitatively, if not quantitatively. Protocols for carrying out array assays are well known to those of skill in the art and need not be described in great detail here. Generally, the sample suspected of comprising the analyte of interest is contacted with an array under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system such as an isotropic or radioactive or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids (or other molecules) that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. References describing methods of using arrays in various applications include U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992—the disclosures of which are herein incorporated by reference.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

An exemplary array is presented in FIG. 4. Array 10 carries multiple probe features 16 disposed across a surface of the substrate 12. The substrate is preferably in the form of a contiguous, substantially planar substrate made of transparent material to facilitate data acquisition scanning there through. Alternatively, the substrate could be scanned from the side which carries features 16. Features 16 (not to scale) are shown disposed in a pattern which defines the array. The extent of the pattern defines a scan region 8.

Array 10 may be set within a housing 14 to provide an array package 30. In which case, substrate 10 is sealed (such as by the use of a suitable adhesive) to housing 14 around a margin 38. Housing 14 is configured such that it and substrate 12, define a chamber into which features 16 of the array face. This chamber is accessible through resilient septa 42, 50 which define normally closed ports of the chamber. An identifier 40, possibly in the form of a bar code, may be affixed to housing 14. The composition of the probe features and material(s) used to produce elements of the array package may vary, but may be as typical in the art.

Further to the discussion above, in using an array that is found acceptable or passes QC measures according to the present invention, the array will typically be exposed to a sample (such as a fluorescently labeled analyte, e.g., protein containing sample) and the array will then be read. Reading of the array may be accomplished by illuminating the array and reading the location and intensity of resulting fluorescence at each feature of the array to detect any binding complexes on the surface of the array.

A scanner that may be used for this purpose is the AGILENT MICROARRAY SCANNER manufactured by Agilent Technologies, Palo Alto, Calif. Other suitable apparatus and methods are described in U.S. Pat. Nos. 5,585,639; 5,760,951; 5,763,870; 6,084, 991; 6,222,664; 6,284,465; 6,329,196; 6,371,370 and 6,406,849—the disclosures of which are herein incorporated by reference. Other suitable scanning devices are commercially available from Axon Instruments in Union City, Calif. and Perkin Elmer of Wellesly, Mass. Analysis of the data, (i.e., collection, reconstruction of image, comparison and interpretation of data) may be employed with associated computer systems and commercially available software, such as GenePix by Axon Instruments, QuantArray by Perkin Elmer or Feature Extraction by Agilent of Palo Alto, Calif.

Yet, the arrays may be read by any method or apparatus other than the foregoing. Other possible reading methods include other optical techniques (for example, detecting chemiluminescent or electroluminescent labels) and electrical techniques (where each feature is provided with an electrode to detect hybridization at that feature in a manner disclosed in U.S. Pat. Nos. 6,251,685 and 6,221,583—the disclosures of which are herein incorporated by reference).

In any case, results from reading an array may be raw results (such as fluorescence intensity readings for each feature in one or more color channels) or may be processed results such as obtained by applying saturation factors to the readings, rejecting a reading for a feature which is above or below a predetermined threshold and/or forming conclusions based on the pattern read from the array (such as whether or not a particular target sequence may have been present in the sample).

The results of the reading (processed or not) may be forwarded (such as by communication) to a remote location if desired, and received there for further use (such as further processing). Stated otherwise, in certain variations, the subject methods may include a step of transmitting data from at least one of the detecting and deriving steps, to a remote location. The data may be transmitted to the remote location for further evaluation and/or use. The same such treatment may be afforded quality control data generated and saved in connection with the diagnostic methodology noted above. In many instances, it may be preferred to pair reading results with quality control information. Any convenient telecommunications means may be employed for transmitting the data, e.g., facsimile, modem, Internet, etc.

Alternatively, or additionally, the data representing array results may be stored on a computer-readable medium of any variety such as noted above or otherwise. Retaining such information may be useful for any of a variety of reasons as will be appreciated by those with skill in the art. The same holds for quality control data (whether it be raw or processed data) produced as described above or otherwise.

Kits

Kits for use in connection with arrays as produced according to the subject invention may also be provided. Such kits preferably include at least a computer readable medium including instructions and programming embodying or adapted to direct at least the quality control or diagnostic functionality discussed above. The instructions may include software installation or setup directions to program an otherwise ordinary scanner so to function as described. The instructions may include directions for operating the program once installed (where it is not automatic and some user input or interface may be desired). Naturally, the instructions may include both types of information.

Providing the software and instructions as a kit may serve a number of purposes. The combination may be packaged and purchased as a means (together with suitable hardware) of upgrading an existing fabrication system that already includes suitable hardware adaptation. Alternatively, the full program or some portion of it (preferably at least such code as defining the subject methodology—alone or in combination with the code already available) and relevant hardware may be provided as system upgrade. Alternately, the combination may be provided in connection with a new array fabrication system in which the software is preloaded on the same. In which case, the instructions may serve as a reference manual (or a part thereof) and the computer readable medium as a backup copy to the preloaded utility.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium (e.g., CD-ROM, diskette, etc.), including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or world wide web. Hardware as required may be shipped or obtained directly by a user. For the software at least, some form of access security or identification protocol may be used to limit access to those entitled to use the subject invention. As with the instructions, the means for obtaining the instructions and/or programming is generally recorded on a suitable recording medium.

It is evident from the above discussion that the above described invention provides an effective and readily applicable way to improve quality control in connection with array fabrication. As such, the subject invention represents a significant contribution to the art.

All publications and patents cited in this specification are herein incorporated by reference as if each individual publication or patent were specifically and individually indicated to be incorporated by reference (except insofar as any may conflict with the present application—in which case the present application prevails). The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. Indeed, the techniques described herein can be applied to any application where discrete spots on a substrate need to be deposited. This includes flat panel displays and the like. All such modifications are intended to be within the scope of the claims appended hereto.

What is claimed is:

1. A method for fabricating a biopolymer array with multiple features, the method comprising:
providing a substrate having a surface;
ejecting reagent drops from an ejection head spaced from said substrate surface, during movement said ejection head and said surface relative to each other, wherein said reagent drops are ejected according to a predetermined pattern onto said surface to produce said array; and
electrically monitoring firing of said ejection head by observation of an electrical signal output by a driver.

2. The method of claim 1, wherein said electrical signal comprises a voltage corresponding to a firing pulse.

3. The method of claim 1, wherein said electrical signal sets an electronic latch.

4. The method of claim 3, wherein said electrical signal voltage is reduced from a voltage of said firing pulse by a voltage divider.

5. The method of claim 2, wherein said electrical signal is processed by an analog-to-digital converter to provide a measure of an amplitude of said firing pulse.

6. The method of claim 1, wherein said ejecting of drops occurs by said driver grounding an individual piezo element in said ejection head.

7. The method of claim 1, wherein said ejection of drops occurs by said driver applying a firing pulse to individual piezo elements in said ejection head.

8. The method of claim 1, wherein said ejecting of drops occurs by said driver grounding an individual resistor element in said ejection head.

9. The method of claim 1, wherein said ejection of drops occurs by said driver applying a firing pulse to individual resistor elements in said ejection head.

10. The method of claim 1, wherein said biopolymers are polyncucleotides.

11. A biopolymer array produced according to the method of claim 1.

12. A method of detecting the presence of an analyte in a sample, said method comprising:
contacting a sample suspected of comprising said analyte with a biopolymer array according to claim 11;
detecting any binding complexes on the surface of the said array to obtain binding complex data; and
determining the presence of said analyte in said sample using said binding complex data.

13. The method of claim 12, wherein said analyte is a nucleic acid.

14. The method of claim 12, wherein said method further comprises a data transmission act in which a result from a reading of said array is transmitted from a first location to a second location.

15. The method of claim 12, wherein said second location is a remote location.

16. A method comprising receiving data representing a result of a reading obtained by the method of 12.

17. A method comprising forwarding data representing a result of a reading an array fabricated by the method of claim 1.

18. A computer-readable medium comprising at least a portion of a program to direct an array fabrication apparatus to perform the method of claim 1.

19. The computer-readable medium of claim 18, wherein the entirety of said program is provided.

20. A kit comprising the computer readable medium of claim 18, in packaged combination with instructions for use with the same.

21. An array fabrication system comprising:
a substrate station to retain a substrate thereon;
an ejection head facing and spaced from said substrate station;
a transport system to move one of the head and substrate station relative to the other;
a controller for the ejection head and transport system; and
a quality control circuit adapted to electrically monitor firing of said ejection head by observation of an electrical signal output by a driver.

22. The system of claim 21, wherein said electrical signal comprises a voltage corresponding to a firing pulse.

23. The system of claim 21, further comprising a gate array comprising a plurality of electronic latches of a gate array, each latch corresponding to a piezo element of said ejection head.

24. The system of claim 23, further comprising a voltage divider to reduce a firing pulse voltage for input to said gate array as said electrical signal.

25. The system of claim 21, wherein a programmable memory is provided to store output of said gate array.

26. The system of claim 21, wherein said ejection head is configured to fire upon grounding an individual element in said ejection head.

27. The system of claim 21, wherein said ejection head is configured to fire upon applying a fire pulse to individual piezo elements in said ejection head.

28. The system of claim 21, wherein said ejection head comprises ejection elements selected from piezoelectric and thermoelectric elements.

29. The system of claim 21, further comprising an analog-to-digital converter connected to receive said electrical signal to provide a firing pulse check.

30. The system of claim 21, further comprising a substrate suitable for producing a biopolymer array, wherein said substrate is mounted to said substrate station.

31. A pulse jet quality control circuit comprising:
at least one ejection head for said pulse jet; and
a means for electrically monitoring firing of each said ejection head by observation of an electrical signal.
